(19) United States
(12) Patent Application Publication
(10) Pub. No.: US 2005/0208611 A1
(43) Pub. Date: Sep. 22, 2005

(54) Process for identifying compounds with fungicide activity based on UMP/CMP kinases from fungi

(76) Inventors: Roman Wieczorke, Dusseldorf (DE); Rudiger Suelmann, Koln (DE); Peter Schreier, Koln (DE); Birgitta Leuthner, Langenfeld (DE)

Correspondence Address: BAYER CROPSCIENCE LP;Patent Department, 100 BAYER ROAD, PITTSBURGH, PA, 15205-9741, US

(21) Appl. No.: 10/996,788

(22) Filed: Nov. 24, 2004

(30) Foreign Application Priority Data
Dec. 3, 2003 (DE) 10356433.0

(51) Int. Cl. C12Q 1/48; C12Q 1/18

(52) U.S. Cl. 435/15; 435/32

(57) ABSTRACT

The invention refers to a process for identifying fungicides, the use of a UMP/CMP kinase for identifying fungicides, as well as nucleic acids coding for UMP/CMP kinases and the polypeptides coded by them.

The invention refers to a process for the identification of fungicides, the use of uridine-5′-monophosphate kinase or cytidine-5′-monophosphate kinase for identifying fungicides, on nucleic acids coding for UMP/CMP kinases and the polypeptides coded by these.

Undesired fungal growth, which leads to considerable damage every year in the agricultural sector, can be controlled by the use of fungicides. Consequently, the demand for fungicides has grown steadily with respect to their effectiveness, cost and above all their environmental compatibility. Thus there is a demand for new substances or substance classes that can be developed into effective, environmentally compatible new fungicides. In general it is typical that such new pharmaceutical lead compounds are searched for in greenhouse tests. However, such tests are labor-intensive and expensive. The number of substances that can be tested in the greenhouse is correspondingly limited. An alternative to such tests is the use of so-called HTS or “high throughput screening”. Here an automated process is used to test a large number of individual substances with respect to their effect on cells, individual gene products or genes. If activity is proven for particular substances, then these can be investigated in conventional screening procedures and developed further where appropriate.

Advantageous targets for fungicides are often looked for in essential biosynthesis pathways. Ideal fungicides are furthermore those substances, which inhibit gene products that play a decisive role in the occurrence of pathogenicity for a fungus.

It was therefore the object of the present invention to identify a suitable new target for potential substances with fungicide activity and to make it available, and to make a process available that enables the identification of modulators of this target, which can then be used as fungicides.

All known eukaryotic uridylate kinases catalyze the efficient phosphorylation of UMP as well as of CMP to the corresponding uridine- and cytidine diphosphates, whereby ATP is used as a phosphate donor. For this reason, uridylate kinases (EC 2.7.4.-) are also known as UMP/CMP kinases (uridine-5′-monophosphate/cytidine-5′-monophosphate kinases) or pyrimidine nucleoside monophosphate kinases (Yan und Tsai, 1999, Adv. Enzymol. Relat. Areas Mol. Biol. 73:103-34). The enzymatic activity of the UMP/CMP kinases can be represented schematically as follows:
ATP+UMP<=>ADP+UDP, or ATP+(d)CMP<=>ADP+(d)CDP

Since all pyrimidines within the cell are derived from UMP, the UMP/CMP kinase is a key enzyme for pyrimidine nucleotide biosynthesis. The reaction catalyzed by the UMP/CMP kinase is an essential step for the supply of UDP for further anabolic metabolic pathways, such as RNA biosynthesis for example or the synthesis of sugar nucleotides such as UDP glucose and UDP galactose (Ma et al., 1990, Journal of Biol. Chem. 256 (31): 19122-19127; Jong et al., 1993, Arch. Biochem. Biophys. 304 (1): 197-204; Zhou et al., 1998, Plant Physiol. 117: 245-254).

Genes for the UMP/CMP kinase were cloned from various fungi: from the yeasts Saccharomyces cerevisiae (Swissprot Accession No.: P15700) and Schizosaccharomyces pombe (Swissprot Accession No.: O59771), from Lentinus edodes (Pir Accession No.: JC6572) as well as from the slime mold Dictyostelium discoideum (Swissprot Accession No.: P20425). In addition to that the UMP/CMP kinase was also isolated from other organisms, for example from Homo sapiens (Swissprot Accession No.: P30085), Mus musculus (Swissprot Accession No.: Q9DBP5), Sus scrofa (Swissprot Accession No.: Q29561), Caenorhabditis elegans (RefSeq Accession No.: NP_{—}496386), Arabidopsis thaliana (Swissprot Accession No.: O04905) and Oryza sativa (Genbank Accession No.: AAF23371). The sequence similarities within the eukaryontic classes are significant (see FIG. 1).

Up to now, UMP/CMP kinases were isolated, expressed, purified and characterized for example from Saccharomyces cerevisiae, Arabidopsis thaliana and Lentinus edodes (Ma et al., 1990, Journal of Biol. Chem. 256 (31): 19122-19127; Zhou et al., 1998, Plant Physiol. 117: 245-254; Kaneko et al., 1998, Gene 211: 259-266).

The object of the present invention was thus to identify new targets for fungicides in fungi, particularly in fungi that are phytopathogenic and to make a procedure available in which inhibitors of such a target or polypeptide can be identified and tested with regard to its fungicide properties. The task was solved by isolating from a phytopathogenic fungus, Ustilago maydis, a nucleic acid coding for a UMP/CMP kinase; the polypeptide coded by it was recovered and a process was made available with which inhibitors of this enzyme can be determined. The inhibitors identified with this process could be applied in vivo against fungi.

### DESCRIPTION OF THE FIGURES

FIG. 1: Sequence alignment of fungal UMP/CMP kinases. Homology between UMP/CMP kinases from various fungi. The frames show areas with an exactly matching sequence (consensus sequence). The amino acids corresponding to the PROSITE motif for the ATPase domain of UMP/CMP kinases are indicated in the figure. S_{—}pombe: Saccharomyces pombe. N_{—}crassa: Neurospora crassa. S_{—}cerevisiae: Saccharomyces cerevisiae. U_{—}maydis: Ustilago maydis.

FIG. 2: SDS-gel chromatography. The results of the heterologous expression of the UMP/CMP kinase in E. coli BL21(DE3)pLysS is shown. The overexpressed His fusion protein has a size of about 33 kilodaltons. A size standard is applied in track “M”. Track 1: Membrane fraction after cell disruption and separation of membrane fraction and cytoplasm fraction by centrifugation, recovered from cells prior to induction with IPTG. Track 2: Membrane fraction, 3.5 hours after induction with 0.05 mM IPTG, cell disruption and separation of membrane fraction and cytoplasm fraction by centrifugation. Track 3: Membrane fraction, 3.5 hours after induction with 0.5 mM IPTG, cell disruption and separation of membrane fraction and cytoplasm fraction by centrifugation. Track 4: Cytoplasm fraction after cell disruption and separation of membrane fraction and cytoplasm fraction by centrifugation, recovered from cells prior to induction with IPTG. Track 5: Cytoplasm fraction of the overexpressed UMP/CMP kinase 3.5 hours after induction with 0.05 mM IPTG, cell disruption and separation of membrane fraction and cytoplasm fraction by centrifugation. Track 6: Cytoplasm fraction of the overexpressed UMP/CMP kinase 3.5 hours after induction with 0.5 mM IPTG, cell disruption and separation of membrane fraction and cytoplasm fraction by centrifugation.

FIG. 3: SDS-gel chromatography. The results of purification of the UMP/CMP kinase from U. maydis using affinity chromatography are shown. The overexpressed His fusion protein has a size of about 33 kilodaltons. A size standard is applied in track “M”. Track 1: Cytoplasm fraction of the overexpressed UMP/CMP kinase 3.5 hours after induction with 0.5 mM IPTG, cell disruption and separation of membrane fraction and cytoplasm fraction by centrifugation. Track 2: Flow-through at the start of application of the cytoplasm fraction to the affinity chromatography column. Track 3: Flow-through at the end of application of the cytoplasm fraction to the affinity chromatography column. Tracks A8 through B3: Elution fractions with purified UMP/CMP kinase.

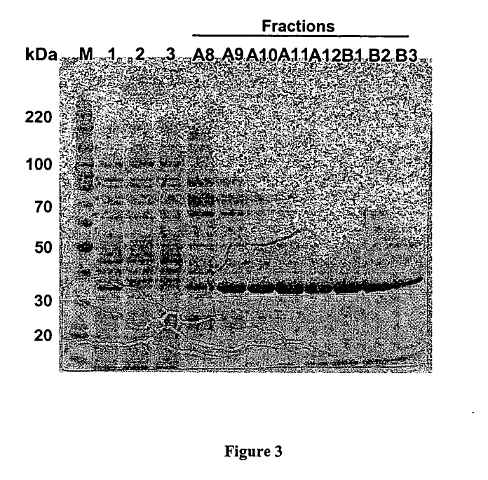

FIG. 4: Graphical representation of the K_{M} value determination for UMP (A) and ATP (B). The measured values are depicted according to Lineweaver and Burk: 1/Vₒ=1/Vₘₐₓ+1/S×(K_{M}/Vₘₐₓ), whereby Vₒ is the initial reaction rate, Vₘₐₓ is the maximum achievable conversion rate and S is the substrate concentration. Vₘₐₓ and K_{M} can then be read as the abscissa and ordinate segment 1/Vₘₐₓ and 1/K_{M} respectively. The K_{M} value is 0.12 mM for UMP and 0.06 mM for ATP.

FIG. 5: Reaction kinetics of the UMP/CMP kinase from Ustilaio madis. The time-dependent conversion of ATP and UMP by the UMP/CMP kinase from Ustilago maydis is shown. The ADP produced in the reaction is measured by the coupled reaction with pyruvate kinase and lactate dehydrogenase. Here the pyruvate produced in the reaction of the pyruvate kinase is converted to lactate by the lactate dehydrogenase with the use of NADH. Thus the measurement of the converted NADH by absorption spectroscopy at 340 nm provides information on the enzymatic activity of the UMP/CMP kinase. As a control, the reaction was measured in the absence of the UMP/CMP kinase.

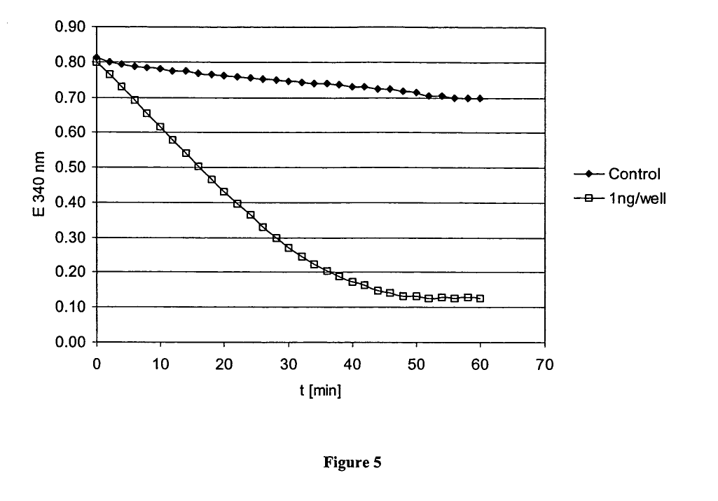

SEQ ID NO:1 Nucleic acid sequence coding for the UMP/CMP kinase from Ustilago maydis.

SEQ ID NO: 2 Amino acid sequence of the UMP/CMP kinase from Ustilago maydis.

Definitions

The term “homology” or “identity” should be understood as the number of matching amino acids (identity) with other proteins, expressed as a percentage. The preferred method of determining identity is by comparing a given sequence with other proteins using computer software. If sequences that are compared to one another have different lengths, the percentage of identity is determined as the number of amino acids that the shorter sequence has in common with the longer sequence. The identity can be routinely determined using known, publicly available computer programs such as ClustalW (Thompson et al., Nucleic Acids Research 22 (1994), 4673-4680). ClustalW, for example, is made publicly available by Julie Thompson (Thompson@EMBL-Heidelberg.DE) and Toby Gibson (Gibson@EMBL-Heidelberg.DE.), European Molecular Biology Laboratory, Meyerhofstrasse 1, D-69117 Heidelberg, Germany. ClustalW can also be downloaded from various Internet pages, including IGBMC (Institut de Génétique et de Biologie Moléculaire et Cellulaire, B.P.163, 67404 Illkirch Cedex, France; ftp://ftp-igbmc.u-strasbg.fr/pub/) and EBI (ftp://ftp.ebi.ac.uk/pub/software/) as well as from all mirrored Internet sites for EBI (European Bioinformatics Institute, Wellcome Trust Genome Campus, Hinxton, Cambridge CB10 1SD, UK). If version 1.8 of ClustalW is used, the following parameters are to be set in order to determine the identity between a given reference protein and other proteins for example: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP. An option for finding similar sequences is the performance of sequence database searches. In this process, one or more sequences are specified as a so-called “query”. This query sequence is then compared with sequences in the selected databases with the help of statistical analysis software. Such database queries (“BLAST searches”) are familiar to specialists and can be performed by various providers. If, for example, such a database query is performed at the NCBI (National Center for Biotechnology Information, http://www.ncbi.nlm.nih.gov/), then the default settings specified for each comparison query should be used. For protein sequence comparisons (“BLASTP”) these settings are as follows: Limit by entrez=not activated; filter=low complexity activated; expect value=10; word size=3; matrix=BLOSUM62; gap costs: existence=11, extension=1. The percentage of identity between the query sequence and the similar sequences found in the databases will be shown in the result of such a query along with other parameters. Therefore an inventive protein in the context of the present invention should be understood as such proteins that show an identity of at least 70%, preferably at least 75%, more preferably at least 80%, even more preferably at least 85% and in particularly preferable at least 90% when at least one of the methods described above is used to determine identity.

The expression “hybridize”, or “hybridization” as it is used herein, describes the procedure whereby a single-strand nucleic acid molecule undergoes base pairing with a complementary strand. In this way, for example, starting from the sequence information given here or derivable herefrom, DNA fragments from phytopathogenic fungi other than Ustilago maydis may be isolated for instance, said fragments coding for UMP/CMP kinases and showing properties the same or similar to one of the inventive UMP/CMP kinases. Short oligonucleotides with a length of about 10 to 50 bp, preferably 15 to 40 bp, are preferred for hybridization, for instance of the conserved areas or other areas that can be characterized by comparisons with other related genes in the manner known to the specialist. However, longer fragments of the inventive nucleic acids or the complete sequences can be used for the hybridization. “Standard conditions”, for example, depending on the nucleic acid, are to be understood as temperatures between 42 and 58° C. in an aqueous buffer solution with a concentration of 0.1 to 5×SSC or additionally the presence of 50% formamide, or for example 42° C. in 5×SSC, 50% formamide. Advantageous conditions for hybridization for DNA:DNA hybrids are at 0.1×SSC and temperatures between about 20° C. and 45° C., preferably between about 30° C. and 45° C. For DNA: with RNA hybrids the preferred conditions for hybridization are at 0.1×SSC and temperatures between about 30° C. and 55° C., preferably between about 42° C. and 55° C. These temperatures for hybridization are exemplary melting point values for a nucleic acid with a length of about 100 nucleotides and a G+C content of 50% in the absence of formamide. The experimental conditions for DNA hybridization are described in relevant textbooks, such as Sambrook et al., “Molecular Cloning”, Cold Spring Harbor Laboratory (1989) for example and can be calculated using formulae known to specialists, dependent for example on the length of the nucleic acids, the type of the hybrids or the G+C content. Conditions for hybridization are calculated approximatively using the following formula: melting point Tm=81.5° C.+16.6{log[c(Na⁺)]}+0.41(% G+C)−(500/n) (Lottspeich, F., Zorbas H. (ed.), “Bioanalytik”, Spektrum Akademischer Verlag, Heidelberg, Berlin (1998)). In this formula, c is the concentration and n the length of the hybridizing sequence segment in base pairs. For a sequence >100 bp, the expression 500/n drops out. Highly stringent washing is performed at a temperature 5 to 15° C. below the Tm and an ionic strength of 15 mM Na⁺ (corresponds to 0.1×SSC). The 1×SSC buffer is composed of the following: 0.15 M NaCl, 15 mM sodium citrate, pH 7.2. Preferred conditions for hybridization are specified as follows: Hybridizing solution: DIG Easy Hyb (Roche, ZZ) hybridization temperature: 40° C. to 70° C., preferably 42 to 55° C. (DNA-DNA) or 50° C. (DNA-RNA). Here, for example, a first washing can be done with 2×SSC, 0.1% SDS 2×5 min at room temperature, followed by a second washing with 1×SSC, 0.1% SDS 2×15 min at 50° C.

The expression “complete UMP/CMP kinase” as it is used herein describes a UMP/CMP kinase that is coded by a complete coding region of a transcription unit starting with the ATG start codon and encompassing all information-bearing exon regions of the gene present in the source organism and coding for UMP/CMP kinase as well as the signals necessary for proper termination of the transcription.

The expression “biological activity of a UMP/CMP kinase” as it is used herein refers to the ability of a polypeptide to catalyze the reaction described above, i.e. the conversion of uridine monophosphate and cytidine monophosphate to uridine diphosphate and cytidine diphosphate.

The expression “active fragment” as it is used herein describes no longercomplete nucleic acids coding for UMP/CMP kinases, which however still code for polypeptides with the biological activity of a UMP/CMP kinase and which can catalyze a characteristic reaction for the UMP/CMP kinase as described above. Such fragments are shorter than the complete nucleic acids coding for UMP/CMP kinase that are described above. Here nucleic acids can have been removed from both the 3′- and/or 5′ ends of the sequence, but parts of the sequence can also have been deleted, i.e. removed, which do not affect the biological activity of the UMP/CMP kinase in a decisively adverse manner. A lower or in some cases even an increased activity, which however still permits the characterization or use of the resultant UMP/CMP kinase fragment, is considered sufficient in the sense of the expression used here. The expression “active fragment” can also refer to the amino acid sequence of the UMP/CMP kinase and then applies analogously to the remarks above for those polypeptides that no longer contain certain parts present in the complete sequence defined above, whereby however the biological activity of the enzyme is not decisively diminished. The fragments can be of various lengths.

The term “UMP/CMP kinase inhibition test”, or “inhibition test” as it is used herein, refers to a process or test, which permits identification of the inhibition of the enzymatic activity of a polypeptide with the activity of a UMP/CMP kinase by one or more chemical compounds (candidate or test compound(s)), by which means the chemical compound can be identified as an inhibitor of the UMP/CMP kinase.

The expression “gene” as it is used herein is the designation for a section from the genome of a cell, which is responsible for the synthesis of a polypeptide chain.

The expression “fungicide” or “fungicidal” as it is used herein refers to chemical compounds suited for combating fungi, particularly phytopathogenic fungi. Such phytopathogenic fungi include but are not limited to:

Plasmodiophoromycetes, Oomycetes, Chytridiomycetes, Zygomycetes, Ascomycetes, Basidiomycetes and Deuteromycetes, for example Pythium species, such as, for example, Pythium ultimum, Phytophthora species, such as, for example, Phytophthora infestans, Pseudoperonospora species, such as, for example, Pseudoperonospora humuli or Pseudoperonospora cubensis, Plasmopara species, such as, for example, Plasmopara viticola, Bremia species, such as, for example, Bremia lactucae, Peronospora species, such as, for example, Peronospora pisi or P. brassicae, Erysiphe species, such as, for example, Erysiphe graminis, Sphaerotheca species, such as, for example, Sphaerotheca fuliginea, Podosphaera species, such as, for example, Podosphaera leucotricha, Venturia species, such as, for example, Venturia inaequalis, Pyrenophora species, such as, for example, Pyrenophora teres or P. graminea (Konidienform: Drechslera, syn: Helminthosporium), Cochliobolus species, such as, for example, Cochliobolus sativus (Konidienform: Drechslera, syn: Helminthosporium), Uromyces species, such as, for example, Uromyces appendiculatus, Puccinia species, such as, for example, Puccinia recondita, Sclerotinia species, such as, for example, Sclerotinia sclerotiorum, Tilletia species, such as, for example, Tilletia caries; Ustilago species, such as, for example, Ustilago nuda or Ustilago avenae, Pellicularia species, such as, for example, Pellicularia sasakii, Pyricularia species, such as, for example, Pyricularia oryzae, Fusarium species, such as, for example, Fusarium culmorum, Botrytis-species, Septoria species, such as, for example, Septoria nodorum, Leptosphaeria species, such as, for example, Leptosphaeria nodoruin, Cercospora species, such as, for example, Cercospora canescens, Alternaria species, such as, for example, Alternaria brassicae or Pseudocercosporella species, such as, for example, Pseudocercosporella herpotrichoides.

Of particular interest are, for example, also Magnaporthe grisea, Cochliobulus heterostrophus, Nectria hematococcus and Phytophtora species.

Fungicidal active substance that are found using the inventive UTMP/CMP kinase can also interact with UMP/CMP kinase from human pathogenic fungus species, whereby the interaction with the various UMP/CMP kinases occurring in these fungi need not always be of the same strength.

The object of the present invention therefore is also a process for identification of antifungal agents, i.e. of inhibitors of the UMP/CMP kinase of human or animal pathogenic fungi, which can be used to produce means of treatment of illnesses caused by human or animal pathogenic fungi.

In this effort, the following human pathogenic fungi are of particular interest, which can cause, among others, the disease patterns mentioned below:

Dermatophytes, such as, for example, Trichophyton spec., Microsporum spec., Epidermophyton floccosum or Keratomyces ajelloi, which can produce fungal infection of the feet (Tinea pedis) for example,

Yeasts, such as, for example, Candida albicans, which causes candidal esophagitis and dermatitis, Candida glabrata, Candida kusei or Cryptococcus neoformans, which can cause pulmonary cryptococcosis and also torulosis,

Molds, such as, for example, Aspergillus fumigatus, A. flavus, A. niger, which cause, for example, bronchopulmonary aspergillosis or flngal sepsis, Mucor spec., Absidia spec., or Rhizopus spec., which cause, for example, zygomycoses (intravasal mycoses), Rhinosporidium seeberi, which causes, for example, chronic granulomatous pharyngitis and tracheitis, Madurella myzetomatis, which causes, for example, subcutaneous myzetomas, Histoplasma capsulatum, which causes, for example, reticuloendothelial cytomycosis and Darling's disease, Coccidioides immitis, which causes, for example, pulmonary coccidioidomycosis and sepsis, Paracoccidioides brasiliensis, which causes, for example, South American blastomycosis, Blastomyces dermatitidis, for example, Gilchrist's disease and North American blastomycosis, Loboa loboi, which causes, for example, Keloid blastomycosis and Lobo's disease and Sporothrix schenckii, which causes, for example, sporotrichosis (granulomatous dermal mycosis).

In the following discussion, the terms “fungicidal” or “fungicide” are used as equivalent to the terms “antifungal” or “antimycotic agents” as well as for the terms “fungicidal” or “fungicides” in the conventional sense, i.e. with reference to phytopathogenic fungi.

Fungicidal active substances that are found using a UMP/CMP kinase isolated from a particular fungus, here for example from Ustilago maydis, can also interact with a UMP/CMP kinase from numerous other fungus species, including phytopathogenic fungi, whereby the interaction with the various UMP/CMP kinases occurring in these fungi need not always be of the same strength. This explains, among other things, the observed selectivity of the substances that have an effect on this enzyme.

The expression “heterologous promoter”, as it is used herein, refers to a promoter that has other properties than the promoter that controls the expression of the respective gene in the source organism.

The expression “competitor”, as it is used herein, refers to the characteristic of the compounds to compete with other, possibly not yet identified compounds for binding to the UMP/CMP kinase and to displace these on the enzyme or be displaced by them.

The expression “agonist”, as it is used herein, refers to a molecule that accelerates or intensifies the activity of the UMP/CMP kinase.

The expression “antagonist”, as it is used herein, refers to a molecule that slows or inhibits the activity of the UMP/CMP kinase.

The expression “modulator”, as it is used herein, is the superordinate term for agonist and antagonist. Modulators can be small organochemical molecules, peptides or antibodies, which bind to the inventive polypeptides or influence their activity. Furthermore, modulators can be small organochemical molecules, peptides or antibodies that bind to a molecule, which in turn binds to the inventive polypeptides, and influences their biological activity in this way. Modulators may constitute natural substrates and ligands or structural or functional mimetics thereof. Preferably however, the expression “modulator”, as it is used herein, involves those molecules which do not constitute natural substrates or ligands.

The expression “inhibitor” or “specific inhibitor”, as it is used herein, denotes a substance, in whose presence a significant, concentration-dependent decrease of the enzymatic activity of the UMP/CMP kinase occurs. Such an inhibitor is preferably “specific”, i.e. it influences the enzymatic activity of the UMP/CMP kinase negatively by specific binding to selected (catalytic or regulatory) sites. In this case the concentration of the specific inhibition is lower than the concentration of an inhibitor that acts non-specifically on the enzyme. Preferably the concentration will be half, particularly preferably one fifth and most particularly preferably a tenth or twentieth of the concentration required for a compound with a non-specific effect.

### DESCRIPTION OF THE INVENTION

Despite comprehensive research on the UMP/CMP kinase, up to now it was unknown that the UMP/CMP kinase in fungi can be a target protein of substances with antifungal activity. Thus in the present invention it is demonstrated for the first time that the UMP/CMP kinase represents a particularly important enzyme for fungi and is therefore particularly well suited to be used as a target protein in the search for additional active substances with improved fungicidal activity.

As part of the effort regarding the present invention, knockout experiments were used to show (Example 1) that the UMP/CMP kinase is an essential enzyme for fungi and therefore can also be a point of attack or “target” for fungicidal active compounds. Thus in the present invention it is demonstrated for the first time that the UMP/CMP kinase represents a particularly important enzyme for fungi and is therefore particularly well suited to be used as a target protein in the search for additional active substances with improved fungicidal activity.

Furthermore, as part of the effort regarding the present invention, a process was developed that is suited for determining the activity of the UMP/CMP kinase as well as the inhibition of this activity in an inhibition test, thereby identifying inhibitors of the enzyme, for example in high throughput screening and ultra-high throughput screening procedures, and testing their fungicidal properties.

As part of the effort regarding the present invention it was found that the UMP/CMP kinases can also be inhibited in vivo by active substances and a fungal organism treated with this active substance can be harmed or killed. Thus the inhibitors of a fungal UMP/CMP kinase can be used as fungicides, particularly in pest management or also as antifungal agents in pharmaceutical indications. In the scope of the present invention it can be shown that inhibition of the UMP/CMP kinase with one of the substances identified in a process according to the invention leads to the treated fungi dying off or being inhibited in growth in synthetic media or on the plant.

UMP/CMP kinases can be isolated from various phytopathogenic or also from human pathogenic or animal pathogenic fungi, for example from the phytopathogenic fungus U. maydis as shown in the present invention. To produce the UMP/CMP kinases from fungi, the gene, recombinant in Escherichia coli for example, can be expressed, and an enzyme preparation can be made from E. coli cells. Preferably UMP/CMP kinases from phytopathogenic fungi are used, in order to identify fungicides which can be used for pest management. If the objective is identification of fungicides or antifungal agents that are to be used in pharmaceutical indications, the use of UMP/CMP kinases from human or animal pathogenic fungi is advisable.

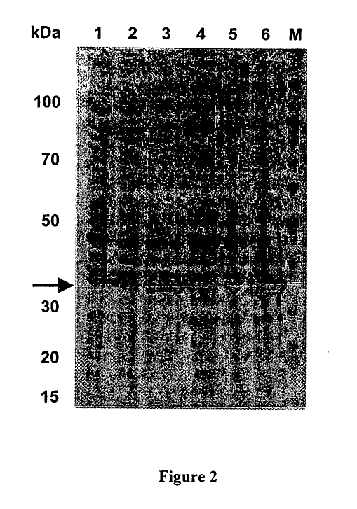

Thus for the expression of the polypeptide UCK coded by uck, the associated ORF was amplified from complete RNA by means of RT-PCR using gene-specific primer according to methods known to the specialist. The corresponding DNA was cloned in the vector pDEST17 (Gateway vector from Invitrogen, provided with an N-terminal His tag). The resulting plasmid pDEST17-UCK contains the complete code sequence of uck in an N-terminal fusion with an HIS tag from the vector. The UCK fusion protein has a calculated mass of 33 kilodaltons (Example 2 and FIG. 2).

The plasmid pDEST17-UCK was then used for recombinant expression of UCK in E. coli BL21 (DE3) pLysS (Example 2).

As already discussed above, the present invention is not limited just to the use of UMP/CMP kinase from Ustilago maydis. In an analogous manner known to the specialist, polypeptides with the activity of a UMP/CMP kinase can be isolated from other fungi, preferably from phytopathogenic fungi, and the polypeptides can then be used in a procedure according to the invention. Preferably the UMP/CMP kinase from

Ustilago maydis is used.

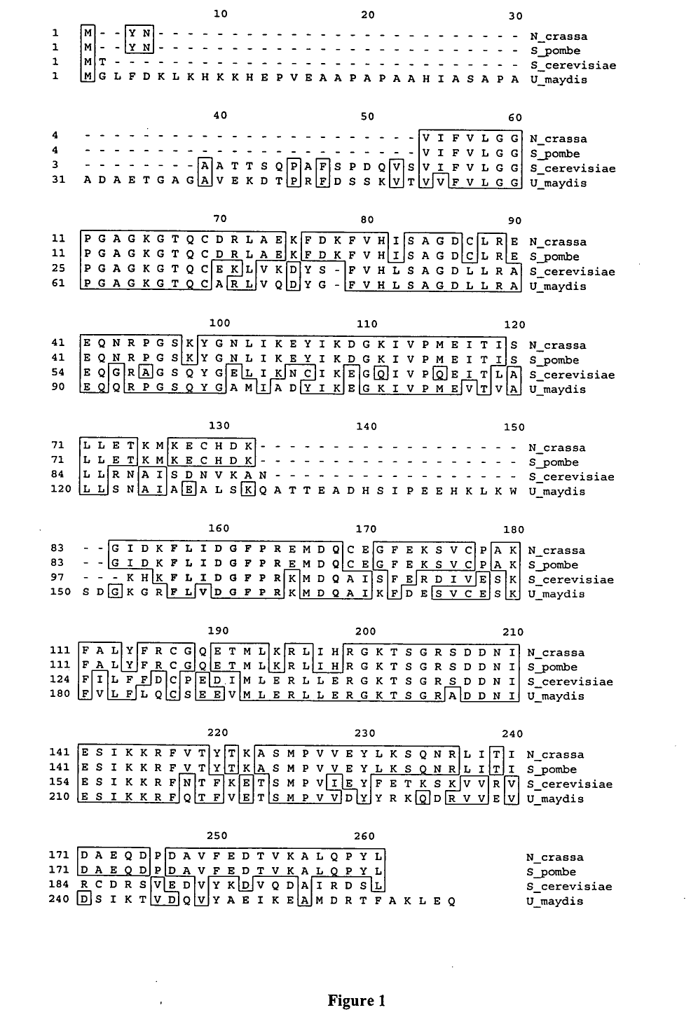

UMP/CMP kinases share homologous regions (see FIG. 1). A conserved region, usually found in ATP-binding enzymes, is characteristic for UMP/CMP kinase. This region includes an aspartic acid residue that is part of the catalytic cleft of the enzyme and that is involved in a salt bridge. It also includes an arginine residue whose modification impairs the activity of the enzyme.

Both of these amino acid residues as well as their sequence environment are a characteristic sequence attribute for UMP/CMP kinases. This sequence motif, characteristic for UMP/CMP kinases, can be represented as a PROSITE motif (Hofmann K., Bucher P., Falquet L., Bairoch A. (1999) “The PROSITE database, its status in 1999”. Nucleic Acids Res. 27, 215). It can be depicted as follows:
[LIVMFYWCA]-[LIVMFYW](2)-D-G-[FYI]-P-R-x(3)-[NQ],
where the moiety D stands for aspartic acid in the active site and G for arginine.

Preferably UMP/CMP kinases with the sequence motif
F-L-[IV]-D-G-F-P-R-x(3)-Q
will be used.

PROSITE enables a pattern to be assigned and thus UMP/CMP kinases to be recognized as such. The single letter code is used to depict the PROSITE motif. The symbol “x” stands for a position, at which any amino acid is accepted. A variable position at which various particular amino acids are accepted, is shown in square brackets “[ . . . ]”, whereby the possible amino acids at this position are listed. Amino acids that are not accepted at a given position, on the other hand, are listed in curly brackets “{ . . . }”. A dash (“-”) separates the individual elements or positions of the motif. If a particular position, such as “x”, is repeated sequentially a number of times, this can be indicated by specifying the number of repetitions in subsequent parentheses, e.g. “x (3)”, which stands for “x-x-x”.

A PROSITE motif thus represents the components of a consensus sequence as well as the intervals between the amino acids involved and is therefore characteristic for a particular enzyme class. With this motif, based on the inventive nucleic acids, further polypeptides from phytopathogenic fungi can be identified or assigned, which belong to the same class as the inventive polypeptide and thus can also be used in the manner according to the invention.

In the case of the UMP/CMP kinase from U. maydis, this motif is present with S. cerevisiae, S. pombe, or N. crassa as well (see FIG. 1).

The aforementioned PROSITE motif or the specific consensus sequence are characteristic for the inventive polypeptides, which can be structurally defined with this consensus sequence and are thus also unambiguously identifiable.

Thus the subject of the present invention also includes polypeptides from phytopathogenic fungi with the biological activity of a UMP/CMP kinase, that contain the aforementioned PROSITE motif [LIVMFYWCA]-[LIVMFYW](2)-D-G-[FYI]-P-R-x(3)-[NQ], with especial preference for the motif F-L-[IV]-D-G-F-P-R-x(3)-Q.

With the homologies present for species-specific nucleic acids coding for UMP/CMP kinases, UMP/CMP kinases from other phytopathogenic fungi can be identified and used, in order to achieve the objective, i.e. they can also be used for identification of inhibitors of a UMP/CMP kinase, which in turn can be used as fungicides in pest management. However, it is also conceivable to use another fungus, which is not phytopathogenic or its UMP/CMP kinase or the sequence coding for it, in order to identify effective fungicidal inhibitors of UMP/CMP kinase. With the sequence specified here according to SEQ ID NO: 1 and possible primers derived therefrom as well as the possible use of the previously given PROSITE motif, it is possible for the specialist to obtain and identify additional nucleic acids coding for UMP/CMP kinases from other (phytopathogenic) fungi, using PCR for example. Such nucleic acids and their use in procedures for identification of fungicidal active substances are considered to be part of the present invention.

Other nucleic acid sequences coding for a UMP/CMP kinase, particularly from phytopathogenic fungi, can be identified using the inventive nucleic acid sequence as well as sequences obtained from other phytopathogenic fungi in accordance with the process described above. Subjects of the present invention are therefore nucleic acids from phytopathogenic fungi, which code for a polypeptide with the enzymatic activity of a UMP/CMP kinase, particularly polypeptides, that contain the motifs described above.

Preferred embodiments of the present invention are nucleic acids from the phytopathogenic fungus species cited above under definitions, which code for a polypeptide with the enzymatic activity of a UMP/CMP kinase.

Particularly preferred embodiments of the present invention are nucleic acids with the SEQ ID NO:1 that code for the UMP/CMP kinase from Ustilago maydis as well as the nucleic acids that code for the polypeptides according to SEQ ID NO:2 or active fragments thereof.

The inventive nucleic acids particularly have to do with single-stranded or double-stranded deoxyribonucleic acids (DNA) or ribonucleic acids (RNA). Preferred embodiments are fragments of genomic DNA, especially cDNAs.

It is particularly preferred that the inventive nucleic acids include a sequence from phytopathogenic fungi, which codes for a polypeptide with the enzymatic activity of a UMP/CMP kinase selected from
a) a sequence according to SEQ ID NO: 1,
b) sequences that code for a polypeptide, that contains the amino acid sequence according to SEQ ID NO: 2,
c) sequences that code for a polypeptide that contains the motif [LIVMFYWCA]-[LIVMFYW](2)-D-G-[FYI]-P-R-x(3)-[NQ], especially preferred are those with the motif F-L-[IV]-D-G-F-P-R-x(3)-Q,
d) sequences that hybridize to the sequences defined under a) and b) at a hybridization temperature of 42 to 65° C., and
e) sequences which show at least an 80% sequence identity with the sequences defined under a) and b), preferred being those with at least 85% and particularly preferred those with at least a 90% identity.

As already discussed above, the present invention is not limited to just UMP/CMP kinase from Ustilago maydis. In an analogous manner known to the specialist, polypeptides with the activity of a UMP/CMP kinase can be isolated from other fungi, preferably from phytopathogenic fungi, and the polypeptides can then be used, for example, in a procedure according to the invention. Preferably the UMP/CMP kinase from Ustilago maydis is used.

Subjects of the present invention are furthermore DNA constructs that contain an inventive nucleic acid and a homologous or heterologous promoter.

The choice of heterologous promoters depends on whether prokaryotic or eukaryotic cells or cell-free systems are used for expression. Examples of heterologous promoters are the 35S promoter from the cauliflower mosaic virus for plant cells, the promoter of the alcohol dehydrogenase for yeast cells, and the T3, T7 or SP6 promoter for prokaryotic cells or cell-free systems. Promoters that are also suitable for expression of UMP/CMP kinases in gram-negative bacteria strains are, for example the cos, tac, trp, tet, lpp, lac, lacIq, T5, gal, trc, ara, 1-PR or 1-PL promoters. Moreover, the promoters ADC1, MFa, AC, P-60, CYC1, GAPDH, TEF, rp28, AOX1 and GAP can be used for expression in yeast strains. For insect cells, for example, the polyhedrin promoter as well as the p10 promoter can be used (Luckow, V. A. and Summers, M. D. (1988) Bio/Techn. 6, 47-55).

Fungal expression systems such as, for example, the Pichia pastoris system should be used preferentially, whereby in this case the transcription is driven by the AOX promoter, which is induced by methanol.

Furthermore, subjects of the present invention are vectors that contain an inventive nucleic acid, an inventive regulatory region or an inventive DNA construct. All phages, plasmids, phagmids, phasmids, cosmids, YACs, BACs, artificial chromosomes or particles, which are suitable for particle bombardment, can be used as vectors.

Preferred vectors are, for example, the commercially available fusion and expression vector pGEX (Pharmacia Biotech Inc), pMAL (New England Biolabs, Beverly, Mass.) and pRIT5 (Pharmacia, Piscataway, N.J.) which contain glutathion S-transferase (GST), maltose binding protein, or protein A, the pTrc vectors (Amann et al., (1988), Gene 69:301-315), the “pKK233-2n” (CLONTECH, Palo Alto, Calif.) and the “pET” and “pBADH” vector series (Stratagene, La Jolla, Calif.). Other advantageous vectors for use in yeast are pYep Secl (Baldari et al. (1987), Embo J. 6:229-234), pMFa (Kurjan and Herskowitz (1982), Cell 30:933-943), pJRY88 (Schultz et al. (1987), Gene 54:113-123), and pYES derivatives, pGAPZ derivatives, pPICZ derivatives, the vectors of the “Pichia Expression Kit” (Invitrogen Corporation, San Diego, Calif.), the p4XXprom. vector series (Mumberg et al., 1995) as well as pSPORT vectors (Life Technologies) for bacteria cells, or Gateway vectors (Life Technologies) for various expression systems in bacterial cells and plants, P. pastoris, S. cerevisiae or insect cells.

Subjects of the present invention also include host cells that contain an inventive nucleic acid, an inventive DNA construct or an inventive vector.

The expression “host cell”, as it is used herein, refers to cells that do not naturally contain the inventive nucleic acids. Suitable host cells include prokaryotic cells, preferably E. coli, but also bacteria of the genusus Erwinia, Flavobacterium, Alcaligenes or cyanobacteria of the genus Synechocystis or Anabena, as well as fungus cells, such as the yeasts Saccharomyces, Candida or Pichia, preferably Saccharomyces cerevisiae or Pichia pastoris, as well as the fungi Aspergillus, Trichoderma, Ashbya, Neurospora, Fusarium, Beauveria, Mortierella or Phythium, or also insects, plants, froschoocytes and mammalian cell lines.

Subjects of the present invention are furthermore polypeptides with the biological activity of a UMP/CMP kinase, which are coded by the inventive nucleic acids.

Preferably the inventive polypeptides contain an amino acid sequence selected from phytopathogenic fungi.
(a) the sequence according to SEQ ID NO:2,
(b) sequences which show at least an 80% sequence identity with the sequence defined under a), preferred being those with at least 85%, particularly preferred those with at least 90% and especially preferred those with 95% identity,
(c) the sequences specified under b), which contain the motif [LIVMFYWCA]-[LIVMFYW](2)-D-G-[FYI]-P-R-x(3)-[NQ], with particular preference for the motif F-L-[IV]-D-G-F-P-R-x(3)-Q, and
(d) fragments of the sequences specified in a) through c), which show the same biological activity as the sequence defined under a).

The expression “polypeptides”, as it is used herein, refers not only to short amino acid chains, which are usually referred to as peptides, oligopeptides or oligomers, but also to longer amino acid chains, which are usually designated as proteins. It covers amino acid chains, which can be modified either by natural processes, such as post-translational processing or by chemical processes that are state-of-the-art. Such modifications can occur at various positions and on a number of occasions in a polypeptide, such as for example on the peptide backbone, on the amino acid side chain, or on the amino- and/or on the carboxylic end group. These include, for example, acetylation, acylation, ADP-ribosylation, amidation, covalent bonding with flavines, heme portions, nucleotides or nucleotide derivatives, lipids or lipid derivatives or phophatidylinositol, cyclization, disulfide bridge formation, demethylation, cystine formation, formylation, gamma-carboxylation, glycosylation, hydroxylation, iodination, methylation, myristoylation, oxidation, proteolytic processing, phosphorylation, selenoylation and transfer-RNA mediated addition of amino acids.

The inventive polypeptides can also be in the form of “mature” proteins or as parts of larger proteins, such as fusion proteins. Furthermore, they can be secretion or “leader” sequences, pro-sequences, sequences that facilitate purification, such as a number of occasions histidine groups, or have additional stabilizing amino acids. The inventive proteins can also be present as they occur naturally in their source organism, from which, for example, they can be isolated directly. Active fragments of a UMP/CMP kinase can also be used in the inventive process, as long as they enable the determination of enzymatic activity of the polypeptide or its inhibition by a candidate compound.

The polypeptides used in the inventive process can have deletions or amino acid substitutions compared to the corresponding regions of naturally occurring UMP/CMP kinases, as long as they at least still show the biological activity of a complete UMP/CMP kinase. Conservative substitutions are preferred. Such conservative substitutions include variations in which an amino acid is replaced by another amino acid from the following group:

1. Low aliphatic, non-polar or slightly polar groups: Ala, Ser, Thr, Pro and Gly;

2. Polar, negatively charged groups and their amides: Asp, Asn, Glu and Gln;

3. Polar, positively charged groups: His, Arg and Lys;

4. Large aliphatic, non-polar groups: Met, Leu, Ele, Val and Cys; and

5. Aromatic groups: Phe, Tyr and Trp.

Purification procedures for the UMP/CMP kinase may be based on preparative electrophoresis, FPLC, BPLC (for example with the use of gel filtration, reverse phase or slightly hydrophobic columns), gel filtration, differential precipitation, ion exchange chromatography or affinity chromatography (see Example 2).

A fast procedure for isolating UMP/CMP kinases synthesized from host cells starts with the expression of a fusion protein, whereby the fusion partner can by purified easily based on affinity. The fusion partner can be an His tag for example (see Example 2). The fusion protein can be purified on a Ni²⁺ column for example. The fusion partner can be removed by partial proteolytic cleavage, for example at the linkers between the fusion partner and the inventive polypeptide to be purified. The linker can be constructed such that it includes target amino acids like arginine groups and lysine groups, which define sites for cleavage by trypsin. Standard cloning procedures with oligonucleotides can be used to generate such linkers.

Other possible purification procedures are based once again on preparative electrophoresis, FPLC, HPLC (for example with the use of gel filtration, reverse phase or slightly hydrophobic columns), gel filtration, differential precipitation, ion exchange chromatography and affinity chromatography.

The expressions “isolation or purification”, as they are used herein, mean that the inventive polypeptides are separated from other proteins or other macromolecules of the cell or tissue. Preferably a composition containing the inventive polypeptides is enriched at least ten-fold and especially preferably at least hundred-fold with respect to the protein content versus a preparation from the host cells.

The inventive polypeptides can also be affinity purified without fusion partners using antibodies that bind to the polypeptides.

The procedure for producing polypeptides with the activity of a UMP/CMP kinase, such as, for example, the polypeptide uck, is characterized by
(a) the cultivation of a host cell, containing at least one expressable nucleic acid sequence coding for a polypeptide from fungi with the biological activity of a UMP/CMP kinase under conditions that ensure the expression of this nucleic acid, or
(b) the expression of an expressable nucleic acid sequence, coding for a polypeptide from fungi with the biological activity of a UMP/CMP kinase in an in vitro system, and
(c) the extraction of the polypeptide from the cell, the culture medium or the in vitro system.

The cells thus obtained, containing the inventive polypeptide or the polypeptide obtained and purified this way, are suitable for use in procedures for identifying modulators and/or inhibitors of the UMP/CMP kinase.

The subject of the current invention is also the use of polypeptides from fungi that carries out at least one biological activity of a UMP/CMP kinase in procedures for identifying inhibitors of a polypeptide from fungi with the activity of a UMP/CMP kinase, whereby the inhibitors of the UMP/CMP kinase can be used as fungicides. The UMP/CMP kinase from Ustilago maydis is particularly preferred for use.

Fungicidal active substances that are found using a UMP/CMP kinase from a particular fungus species can also interact with UMP/CMP kinases from other fungus species, whereby the interaction with the various UMP/CMP kinases occurring in these fungi need not always be of the same strength. This explains, among other things, the selectivity of effective substances. The use of active substances found with a specific UMP/CMP kinase as fungicides for other fungus species as well functions on the basis that UMP/CMP kinases from various fungus species are very similar and show pronounced homology in large regions. Thus it is clear from FIG. 1 that such a homology covering extensive sequence segments between S. cerevisiae, N. crassa, S. pombe and U. maydis exists and therefore the activity of substances found using the UMP/CMP kinase from U. maydis does not remain limited to U. maydis.

Therefore, the subject of the present invention is also a procedure for the identification of fungicides by testing potential inhibitors and/or modulators of the enzymatic activity of the UMP/CMP kinase (“candidate compounds” or “test compounds”) in a UMP/CMP kinase inhibition test.

Procedures that are suited for identifying modulators, particularly inhibitors or antagonists of the inventive polypeptides, are generally based on determining the activity and/or biological functionality of the polypeptide. In principle, both procedures affecting whole cells (in vivo procedures) as well as those based on the use of polypeptides isolated from the cells are applicable, the latter available in purified or partially purified form or even as a raw extract. These cell-free in vitro procedures can be used on a laboratory scale, just like in vivo procedures, but preferably also in high throughput screening or ultra-high throughput screening procedures. Subsequent to the in vivo or in vitro identification of modulators of the polypeptide, tests can be performed on fungus cultures, in order to test the fungicidal effectiveness of the compounds found.

Many test systems with the purpose of testing compounds and natural extracts are designed with a preference for high throughput, in order to maximize the number of substances investigated in a given time period. Test systems based on cell-free procedures need purified or partially purified protein. They are suited for initial screening, the primary purpose of which is to detect a possible influence of a substance on the target protein. If such screening has taken place and one or more compounds, extracts, etc. are found, the effect of such compounds can be studied in a more targeted fashion in the laboratory. Thus in a first step, the inhibition or activation of the inventive polypeptide can be re-tested in vitro, in order to test the effectiveness of the compound subsequently on the target organism, in this case one or more phytopathogenic fungi. If necessary, the compound can then be used as a starting point for the continued search and development of fungicidal compounds based on the original structure, but optimized, for example, with respect to effectiveness, toxicity or selectivity.

In order to find modulators, for example, a synthetic reaction mixture (such as products of the in vitro transcription) or a cellular component, such as a membrane, a cell compartment or any other preparation containing the inventive polypeptides can be incubated with a labeled substrate or ligand of the polypeptides if need be in the presence and absence of a candidate molecule. The ability of the candidate molecule to inhibit the activity of the inventive polypeptides is recognizable, for example, by reduced binding of the possibly labeled ligand or by reduced conversion of the possibly labeled substrate. Molecules that inhibit the biological activity of the inventive polypeptides are good antagonists.

The detection of the biological activity of the inventive polypeptides can be improved by a so-called reporter system. Reporter systems in this regard include, but are not limited to colorimetrically or fluorometrically detectable substrates that are converted to a product or a reporter gene that responds to changes of activity or the expression of the inventive polypeptides or other known binding tests.

A further example of a procedure with which modulators of the inventive polypeptides can be found is a displacement test, in which, under suitable conditions, one brings the inventive polypeptides and a potential modulator in contact with a molecule, that is known to bind to the inventive polypeptides, such as a natural substrate or ligands or a substrate or ligand mimetic. Thee inventive polypeptides themselves can be labeled, for example fluorometrically or colorimetrically, so that the number of polypeptides that are bound to a ligand or that have participated in a conversion, can be measured exactly. However, the binding can also be monitored using the possibly labeled substrate, ligands or substrate analogs. The effectiveness of antagonists can be measured this way.

Another option for identification of substances that modulate the activity of the inventive polypeptides is thus also the “scintillation proximal assay” (SPA, see EP 015473). This test system uses the interaction of a polypeptide (such as UMP/CMP kinase from U. maydis) with a radiolabeled ligand or substrate. Here the polypeptide is bound to microspheres or beads that are tagged with scintillating molecules. In the course of radioactive decay, the scintillating substance in the spheres becomes excited by the subatomic particles of the radioactive marker and emits a detectable photon. The test conditions are optimized so that only the particles originating from the ligand result in a signal, i.e. emitted from one of the ligands bound to the inventive polypeptide.

Effects such as cell toxicity are generally ignored in these in vitro systems. The test systems check both inhibitive or suppressive effects of the substances as well as stimulative effects. The effectiveness of a substance can be tested by concentration-dependent test series. Control samples without test substances or without the enzyme can be examined to evaluate the effects.

The host cells available based on the present invention, which contain nucleic acids coding for an inventive UMP/CMP kinase, enable the development of test systems based on cells, which can be used for the identification of substances that modulate the activity of the inventive polypeptides.

Another option for identification of inhibitors of the inventive polypeptide is based on in vivo or cell-based procedures, which are based principally on the following steps: (1) Production of transgenic organisms, which after transformation with an inventive nucleic acid are able to express a UMP/CMP kinase, (2) application of a test compound to the organism from step (1) and as a control to an analogous, untransformed organism, (3) determining the growth or viability of the transgenic and an untransformed organism after application of the test compound from step (2), and (4) selection of test compounds, which induce reduced growth compared with the growth of the transgenic organism, reduced viability and/or decreased pathogenicity of the non-transgenic organism. An analogous, untransformed organism is to be understood as an organism used as the starting organism in step (1). The transformation can take place with an inventive vector or the inventive nucleic acid itself. Fungi are preferred as organisms that are transformed with the inventive nucleic acid or vector; particularly preferred are the phytopathogenic fungi initially mentioned.

The modulators to be identified will preferably be small organochemical compounds.

A procedure for identification of a compound that modulates the activity of a UMP/CMP kinase from fungi and can be used as a fungicide in pest management preferably is constituted such that
a) an inventive polypeptide or a host cell containing this polypeptide is brought in contact with a chemical compound or with a mixture of chemical compounds under conditions that permit the interaction of a chemical compound with the polypeptide,
b) the activity of the inventive polypeptide in the absence of a chemical compound is compared with the activity of the inventive polypeptide in the presence of a chemical compound or a mixture of chemical compounds, and
c) the chemical compound that specifically modulates the activity of the inventive polypeptide is determined, and if necessary
d) the fungicidal effect of the defined compound is tested in vivo.

Particularly preferred in this regard is the choice of a compound that specifically inhibits the activity of the inventive polypeptide. The term “activity” as it is used here refers to the biological activity of the inventive polypeptide.

A preferred procedure makes use of the fact that ADP is released in the reaction of the UMP/CMP kinase. The activity and/or decrease or increase in activity of the inventive polypeptide can therefore occur by coupling this reaction with the enzymatic conversion of the ADP by the pyruvate kinase and the subsequent enzymatic conversion by the lactate dehydrogenase of the pyruvate produced. This makes use of the fact that the pyruvate produced in the reaction of the pyruvate kinase is converted to lactate by the lactate dehydrogenase (LDH) with the consumption of NADH. The three enzymatic reactions coupled with one another can be depicted as follows, for example:
I. ATP+UMP<=>ADP+UDP (UMP/CMP kinase)
II. phosphoenolpyruvate+ADP+H⁺<=>pyruvate+ATP (pyruvate kinase)
III. pyruvate+NADH+H⁺<=>lactate+NAD⁺ (lactate dehydrogenase)

The co-substrate NADH consumed in the reaction of the LDH has an extinction maximum at 340 nm. The verification of converted NADH by measuring the decrease in absorption at 340 nm or the decrease in fluorescence at 340 nm (emission at 465 nm) therefore provides information on the enzymatic activity of the UMP/CMP kinase.

Lower or inhibited activity of the inventive polypeptide, which leads to reduced synthesis of ADP, leads in the end to lower consumption of NADH. A lesser decrease of the NADH concentration in the presence of a test compound in the inhibition test, compared with a sample without the test compound, indicates inhibition of the enzymatic activity of the UMP/CMP kinase. Conversely, a greater decrease in the NADH concentration in the presence of a test compound in comparison to the control means that the test compound has a stimulating effect on the enzymatic activity of the UMP/CMP kinase.

The measurement can also be carried out in the more usual formats for high throughput screening or UHTS assays, for example on microtiter plates, in which, for example, a total volume of 5 to 50 μl per sample or per well are provided and the individual components are present in the desired final concentration (see Example 3). Here the compound (candidate or test compound) to be tested, which potentially inhibits or stimulates the activity of the enzyme, is, for example, provided in a suitable concentration in test buffer containing ATP, UMP and/or CMP, PEP, NADH as well as the helper enzymes, pyruvate kinase and lactate dehydrogenase. Then the inventive polypeptide in test buffer is added, thereby starting the reaction. The sample is then incubated, for example for 30 minutes at a suitable temperature, and the decrease in absorption is measured at 340 nm for instance.

A further measurement is made with a corresponding sample, which, however, has not had a candidate molecule added and which has not had an inventive polypeptide added (negative control). A further measurement is made in turn in the absence of a candidate molecule, but in the presence of the inventive polypeptide (positive control). Negative and positive controls thus provide the comparison values for the samples in the presence of a candidate molecule.

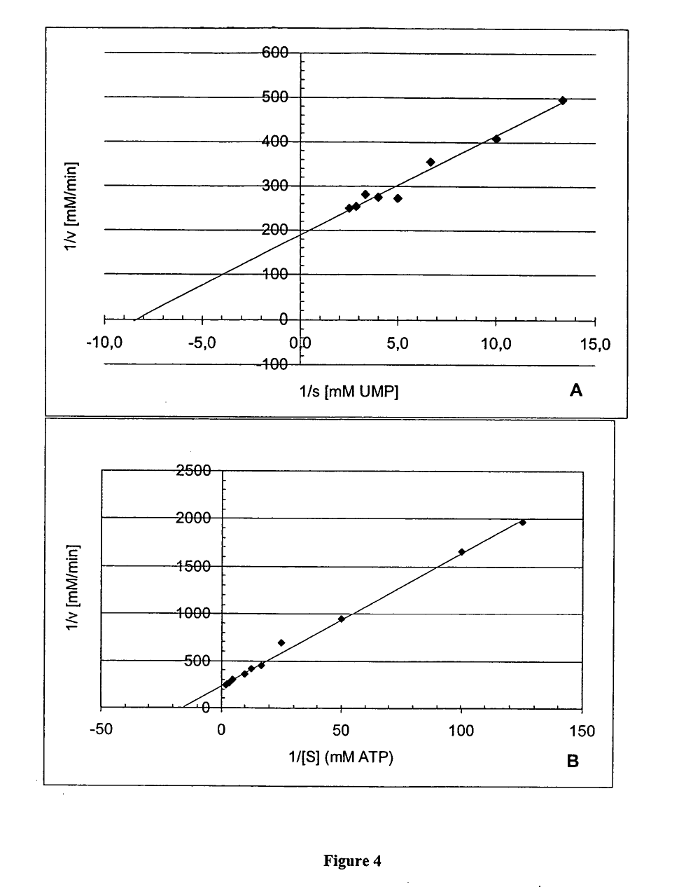

In order to determine the optimum conditions for a process for identifying inhibitors of the UMP/CMP kinase or for determining the activity of the inventive polypeptides, it can be advantageous to determine the respective Kₘ value of the inventive polypeptide used. This gives information on the preferred concentration of the substrate or substrates. In the case of the UMP/CMP kinase from U. maydis a Kₘ of 0.12 mM for UMP and a Kₘ of 0.06 mM for ATP were determined (see FIGS. 4A and 4B).

Using the procedure previously described as an example, compounds can be identified that inhibit the fungal UMP/CMP kinase.

In addition to the exemplary procedure for the determination of the enzymatic activity of a UMP/CMP kinase or the inhibition of this activity and for the identification of fungicides, other procedures, for example known procedures for the determination of der enzymatic activity of UMP/CMP kinases, can be used, as long as these procedures permit a change of this activity in the presence of a potential inhibitor or activator to be recognized.

In the scope of the present invention it can also be tested whether inhibitors of an inventive UMP/CMP kinase, identified using an inventive procedure, are suitable, alone or in an appropriate formulation, for harming or killing fungi. The inhibitors of fungal UMP/CMP kinases can then be used as fungicides.

For this purpose, for example, a methanol solution of the active substance to test, mixed with the emulsifier PS16, was pipetted into the wells of microtiter plates. After the solvent is evaporated, 200 μl of potato dextrose medium are added to each well. The medium is spiked beforehand with a suitable concentration of spores or mycelia of the fungi to be tested. The resulting concentrations of the active substance are 0.05, 0.5, 5 and 50 ppm. The resulting concentration of the emulsifier is 300 ppm.

The plates are subsequently incubated at 20° C. for 3 to 5 days on a shaker until sufficient growth is observable in the untreated control. Photometric evaluation is carried out at a wavelength of 620 nm. The dose to cause a 50% inhibition of the fungal growth with respect to untreated control (ED₅₀) is calculated from the measured data of the various concentrations.

Compounds identified using an inventive process and which, based on the inhibition of fungal UMP/CMP kinases, exhibit fungicidal activity, can thus be used for the production of fungicidal materials.

The identified active substances can be compounded, depending on their particular physical and/or chemical properties, in typical formulations, such as solutions, emulsions, suspensions, powders, foams, pastes, granulates, aerosols, microencapsulations in polymeric materials and in coatings for seeds, as well as ultra-low volume cold and warm fog formulations.

These formulations are produced according to known methods, such as by mixing the active substances with extenders, i.e. liquid solvents, pressurized, liquified gases and/or solid carrier materials, if necessary with the use of surface-active materials, i.e. emulsifiers and/or dispersing agents and/or foam-producing materials. In the case that water is used as an extender, organic solvents can also be used as solubility aids for example. The following liquid solvents are the main ones to be considered: aromatics, such as xylene, toluene or alkyl naphthalene, chlorinated aromatics or chlorinated aliphatic hydrocarbons, such as chlorobenzenes, chloroethylenes or methylene chloride, aliphatic hydrocarbons, such as cyclohexane or paraffins, such as petroleum fractions, alcohols, such as butanol or glycol as well as their ethers and esters, ketones, such as acetone, methyl ethyl ketone, methyl isobutyl ketone or cyclohexanone, strongly polar solvents, such as dimethyl formamide and dimethyl sulfoxide, as well as water. Liquified gas extenders or carriers are liquids that are gases at normal temperature and normal pressure, for example aerosol propellants, such as halogenated hydrocarbons, as well as butane, propane, nitrogen and carbon dioxide. Solid carriers to be considered are: for example, natural mineral powders, such as kaolines, clays, talc, chalk, quartz, attapulgite, montmorillonite or diatomaceous earth and synthetic mineral powders, such as highly disperse silica, aluminum oxide and silicates. Solid carriers to be considered for granulates are: for example, crushed and fractionated natural minerals such as calcite, marble, pumice, sepiolite, dolomite as well as synthetic granulates from inorganic and organic powders as well as granulates from organic material such as sawdust, coconut shells, corn cobs and tobacco stalks. Emulsifiers and/or foam-producing materials to be considered are: for example non-ionizable and anionic emulsifiers, such as polyoxyethylene-fatty acid esters, polyoxyethylene-fatty alcohol ethers, such as alkyl aryl polyglycol ethers, alkyl sulfonates, alkyl sulfates, aryl sulfonates as well as protein hydrolysates. The following may be used as dispersing agents: lignin, sulfite waste liquors and methyl cellulose for example.

Formulations can also include bonding agents like carboxymethyl cellulose, natural and synthetic polymers in powdered, granular or latex-like form, such as gum arabic, polyvinyl alcohol, polyvinyl acetate, as well as natural phospholipids, such as cephalins and lecithins, and synthetic phospholipids. Other additives can be mineral and vegetable oils.

Dyes, such as inorganic pigments, for example iron oxide, titanium oxide, Prussian blue and organic dyes, such as alizarin, azo and metal phthalocyanine dyes and trace elements, such as salts of iron, manganese, boron, copper, cobalt, molybdenum and zinc can be used.

The formulations usually contain between 0.1 and 95 weight percent of the active substance, preferably between 0.5 and 90%.

The inventive active substances can be used as such or in their formulations, also mixed with known fungicides, bactericides, Aacaricides, nematicides or insecticides, in order, for example, to increase the spectrum of effectiveness or prevent the development of resistances. In many cases, synergistic effects are achieved, i.e. the effectiveness of the mixture is greater than the effectiveness of the individual components.

In the use of the inventive compounds as fungicides, the application rates can be varied over wide ranges.

All plants and plant parts can be treated in accordance with the invention. As plants in this context, all plants and plant populations are meant, such as desirable wild plants and undesired wild plants (weeds) or cultured plants (including natural occurring cultured plants). Cultured plants can be plants that can be obtained through conventional breeding and optimization methods or through methods of biotechnology and gene technology or a combination of these methods, including transgenic plants and including those plant types which may be eligible or not be eligible for plant variety protection under law. Plant parts should be understood as all above-ground and subterranean parts and organs of plants, such as sprout, leaf, flower and root, whereby for example leaves, needles, stalks, stems, flowers, fruiting bodies, fruits and seeds as well as roots, tubers and rhizomes are listed. Plant parts also include harvest product as well as vegetative and generative propagation material, such as cuttings, tubers, rhizomes, scions and seeds.

The treatment of plants and plant parts with the active substances in accordance with the invention is done directly or by acting on their environment, habitat or storage space by conventional treatment methods, such as by immersion, spraying, vapor exposure, fogging, scattering, spreading and by propagation material, particularly seeds, furthermore by single or multi-layered coverage.

The following examples illustrate various aspects of the present invention and are not limiting in scope.

### EXAMPLES

### Example 1

Production of uck1 Knockout Mutants in U. maydis

Cultivation of U. maydis

The strains were grown at 28° C. in PD, YEPS or suitable minimal media (Holliday, 1974; Tsukada et al., 1988). The formation of dicaryotic filaments was observed after strains were spotted on PD plates with 1% charcoal (Holliday, 1974). Pathogenicity tests were performed as described (Gillessen et al., 1992). Overnight cultures of the strains were re-suspended at a concentration of 4×10⁷ cells and young corn plants (Gaspé Flint) were injected or spotted. For each strain, at least 25 plants were infected and tumors that occurred were examined after 14 to 21 days.

Synthesis of the Knockout Cassette

Standard molecular biology methods were used (Sambrook et al., 1989). In order to produce uck null mutants, the 5′ and 3′ flank of the uck gene was amplified using PCR. Genomic DNA of the strain UM518 was used as a template. For the 5′ flank (1418BP) the primers LB2 (sequence 5′-cacggcctgagtggccccgagatcaaggaggctatggatc-3′) and p15 (5′-ccgagcgtggattcgagttcc-3′) were used. For the 3′-flank (1393BP) the primers RB1 (5′-gtgggccatctaggccccttctcaacggcaccagcacc-3′) and p13 (5′-ccatcgttgccctaggcactcgcc-3′) were used. The restriction sites Sfi I (a) and Sfi I (b) were introduced with the primers LB2 and RB1. The amplicons were cleaved with Sfi I and ligated with the 1884 bp Sfi I fragment isolated from the vector pBS (hygromycin B cassette). A PCR with the primers LB1 (5′-cctacgcagacgatccaccacc-3′) and RB2 (5′-cgtggatcgcctagaaaaagcgcc-3′) was used to amplify the 3851 bp uck1 knockout cassette, which was used in the subsequent transformation (Kämper and Schreier, 2001).

Production of Protoplasts of U. maydis

50 ml of a culture in YEPS medium were grown at 28° C. to a cell density of about 5×10⁷/ml (OD 0.6 through 1.0) and then centrifuged for 7 min at 2500 g (Heraeus, 3500 rpm) in a 50 ml Falcon tube. The cell pellet was re-suspended in 25 ml SCS buffer (20 mM sodium citrate pH 5.8, 1.0 M sorbitol, (mix 20 mM sodium citrate/1.0 M sorbitol and 20 mM citric acid/1.0 M sorbitol and adjust to pH 5.8 using a pH meter)), centrifuged again for 7 min at 2500 g (3500 rpm) and the pellet re-suspended in 2 ml SCS buffer, pH 5.8, with 2.5 mg/ml Novozym 234. Protoplasting took place at room temperature and was monitored every 5 minutes with a microscope. The protoplasts were then mixed with 10 ml SCS buffer and centrifuged at 1100 g (2300 rpm) for 10 minutes. The supernatant was discarded. The pellet was carefully re-suspended in 10 ml SCS buffer and centrifuged. The washing procedure with SCS buffer was repeated twice and the pellet was washed in 10 ml STC buffer. Finally, the pellet was re-suspended in 500 μl cold STC buffer (10 mm Tris-HCL pH 7.5, 1.0 M sorbitol, 100 mM CaCl₂) and placed on ice. Aliquots can be stored at −80° C. for several months.

Transformation of U. maydis

The transformation of a diploid strain of U. maydis was carried out as described by Schulz et al., 1990. The isolation of genomic U. maydis DNA success was carried out as described by Hoffmann and Winston 1987 or following the method from Qiagen (DNeasy Kit).

For the transformation a maximum of 10 μl DNA (optimally 3-5 μg) were transferred to a 2 ml Eppendorf reaction tube and 1 μl heparin (15 μg/μl) (SIGMA H3125) and 50 μl protoplasts were added. After an incubation period of 10 min on ice, 500 μl of 40% (w/w) PEG3350 (SIGMA P3640) in STC (sterile filtered) were added, mixed carefully with the protoplasts suspension and incubated for 15 minutes on ice. Plating was done on gradient plates (bottom agar: 10 ml YEPS-1.5% agar-1 M sorbitol with antibiotic; shortly before plating the bottom agar layer was coated with 10 ml YEPS-1.5% agar-1M sorbitol, the protoplasts spread and the plates were incubated for 3-4 days at 28° C.

Verification of the homologous recombination in a genomic locus of uck took place by standard methods (PCR or Southern analysis) using isolated genomic DNA. In this way it was shown that the integration of the uck knockout cassette had occurred in a genomic uck locus and thus a wild type copy of the uck gene was replaced, while the second copy remained intact in this diploid strain. The heterozygotic uck mutants were subsequently used in the pathogenicity test (Gillessen et al., 1992).

Spore Analysis of U. maydis

Spores were isolated from the tumors induced in the pathogenicity test. Then the sporidia that evolved were isolated and phenotypically and genotypicallycharacterized. The phenotypic analysis was performed using growth experiments on suitable full or minimal media (Holliday, 1974; Tsukada et al., 1988). This revealed that none of the 48 sporidia analyzed grew under selective conditions. This was a first indication that the phenotype of the uck null mutant was lethal. The genotypic studies were carried out by Southern- or PCR-based analysis of the sporidia. Here it was found that, among the 96 sporidia analyzed, no viable, haploid strain could be identified in which only the uck gene was replaced by the uck knockout cassette. In the cases where the uck knockout cassette was homologously integrated in the genomic locus of uck, an ectopic copy of the gene was also found. These results led to the conclusion that the knockout of the uck gene in Ustilago maydis leads to a lethal phenotype.

### Example 2

Cloning, Expression and Purification of uck or UCK from Ustilago maydis

For cloning uck, the ORF was amplified by means of RT-PCR from complete mRNA of Ustilago maydis using gene-specific primers. The corresponding DNA, an amplicon of 798 bp length, was cloned in the vector pDEST17 (vector of the “Gateway” system from Invitrogen). The resulting plasmid pDEST17-UCK contains the complete code sequence of uck in an N-terminal fusion with the His tag that is part of the vector pDEST17. The UCK fusion protein has a calculated mass of 33 kilodaltons.

For the heterologous expression of the UCK protein, the plasmid pDEST17-UCK was transformed in the BL21(DE3)pLysS strain of E. coli. 50 ml of selection medium (LB medium with 50 μg/ml ampicillin and 34 μg/ml chloramphenicol) were inoculated with the E. coli transformants and grown over night at 30° C. with shaking. 500 ml of selection medium (LB medium with 50 μg/ml ampicillin and 34 μg/ml chloramphenicol) were inoculated with an OD₆₀₀ of 0.1 from this preculture and incubated at 30° C. with shaking. The induction of the gene expression occurred at an OD₆₀₀ of 0.8 to 1.0 by addition of IPTG up to a final concentration of 1 mM. After an induction period of 3.5 hours at 30° C., the cells were harvested by centrifugation, and the cell pellet was stored at −80° C.

For the cell disruption, the pellet was thawed on ice, re-suspended in 4 ml binding buffer (0.05 M NaPO₄, 0.5 M NaCl, 0.02 M imidazole, pH 7.4) and subsequently disrupted for 3 min on ice by sonification. The soluble cytoplasm fraction was obtained by centrifugation for 15 minutes at 4° C. with 10,000 g, and after sterile filtration through a 0.45 μM filter, it was used for purification of the expressed proteins. Purification was carried out by affinity chromatography using the ÄKTAexplorer FPLC system from Amersham using of HiTrap Chelating HP 5 ml columns according to the manufacturer's specifications. The fractions of the protein eluted on the column (elution buffer: 0.05 M NaPO₄, 0.5 M NaCl, 0.M M imidazole, 20% glycerine, pH 7.4) were combined and stored at −80° C.

### Example 3

Identification of Modulators of the UMP/CMP Kinase in 384 Well MTP in a Coupled Assay

384 well microtiter plates from Greiner were used for identifying modulators of the UMP/CMP kinase.

The negative control was pipetted into columns ones and two. This was made of 5 μl 5% DMSO, 20 μl substrate solution (50 mM KPO₄, pH 7.6; 0.75 mM NADH; 0.375 mM ATP; 0.75 mM PEP; 0.75 mM UMP; 25 mM DTT; 12.5 mM MgCl₂; 0.01% Tween20) and 25 μl enzyme solution (50 mm KPO₄, pH 7.6; 0.1% BSA; 8 U/ml pyruvate kinase; 16 U/ml lactate dehydrogenase).

The positive control was pipetted into columns three and four. This was made of 5 μl 5% DMSO, 20 μl substrate solution (50 mM KPO₄, pH 7.6; 0.75 mM NADH; 0.375 mM ATP; 0.75 mM PEP; 0.75 mM UMP; 25 mM DTT; 12.5 mM MgCl₂; 0.01% Tween20) and 25 μl enzyme solution (50 mm KPO₄, pH 7.6; 0.1% BSA; 8 U/ml pyruvate kinase; 16 U/ml lactate dehydrogenase; 5.1; 0.04 μg/ml UMP/CMP kinase).

A test substance was placed in the remaining columns in a concentration of 10 μM in DMSO, with 5 μl of the test buffers used for dilution. After addition of 20 μl substrate solution (50 mM KPO₄, pH 7.6; 0.75 mM NADH; 0.375 mM ATP; 0.75 mM PEP; 0.75 mM UMP; 25 mM DTT; 12.5 mM MgCl₂; 0.01% Tween20), 25 μl enzyme solution (50 mm KPO₄, pH 7.6; 0.1% BSA; 8 U/ml pyruvate kinase; 16 U/ml lactate dehydrogenase; 5.1; 0.04 μg/ml UMP/CMP kinase) were added to start the reaction.

This was followed by incubation at room temperature for 30 minutes. The NADH converted during the reaction was subsequently measured by determining the decrease in absorption at 340 nm in a SPECTRAFluor Plus from Tecan suited for microtiter plates.

### Example 4

Verification of the Fungicidal Activity of the Identified Inhibitors of the UMP/CMP Kinase

A methanol solution of the active substance to test, mixed with the emulsifier PS16, was pipetted into the wells of microtiter plates. After the solvent is evaporated, 200 μl of potato dextrose medium are added to each well. The medium is spiked beforehand with a suitable concentration of spores or mycelia of the fungi to be tested. The resulting concentrations of the active substance are 0.05, 0.5, 5 and 50 ppm. The resulting concentration of the emulsifier is 300 ppm.

The plates are subsequently incubated at 20° C. for 3 to 5 days on a shaker until sufficient growth is observable in the untreated control.

Photometric evaluation is carried out at a wavelength of 620 nm. The dose to cause a 50% inhibition of the flngal growth with respect to untreated control (ED₅₀) is calculated from the measured data of the various concentrations.

References

1. Amann et al., (1988), Tightly regulated tac promoter vectors useful for the expression of unfused and fused proteins in Escherichia coli. Gene 69:301-315.
2. Baldari et al. (1987), A novel leader peptide which allows efficient secretion of a fragment of human interleukin 1 beta in Saccharomyces cerevisiae. Embo J. 6:229-234.
3. Gillissen, B., Bergemann, J., Sandmann, C. Schroeer, M., Bölker, M., and Kahmann, R. (1992), A two-component regulatory system for self/nonself recognition in Ustilago maydis. Cell, 68: 647-657.
4. Hoffman, Charles S.; Winston, Fred. A ten-minute DNA preparation from yeast efficiently releases autonomous plasmids for transformation of Escherichia coli. Gene (1987), 57(2-3), 267-72.
5. Hofmann K., Bucher P., Falquet L., Bairoch A. (1999) “The PROSITE database, its status in 1999”. Nucleic Acids Res. 27, 215.
6. Holliday, R. (1974), Ustilago maydis. In King, R. C. (ed), Handbook of Genetics. Plenum, New York, pp. 575-595.
7. Jong, A. et al. (1993), Characteristics, substrate analysis, and intracellular location of Saccharomyces cerevisiae UMP kinase. Arch. Biochem. Biophys. 304 (1): 197-204.
8. Kámper und Schreier (2001), Verfahren zur Herstellung von Deletionsmutanten; German patent application no. 10 133 926.3.
9. Kaneko, S. et al. (1998), Cloning, sequence analysis and expression of the basidiomycete Lentinus edodes gene uck1, encoding UMP-CMP kinase, the homologue of Saccharomyces cerevisae URA6 gene. Gene 211: 259-266.
10. Kurjan and Herskowitz (1982), Structure of a yeast pheromone gene (MF alpha): a putative alpha-factor precursor contains four tandem copies of mature alpha-factor. Cell 30:933-943.
11. Lottspeich, F., Zorbas H. (ed.). (1998). Bioanalytik. Spektrum Akademischer Verlag, Heidelberg, Berlin.
12. Luckow, V. A. and Summers, M. D. (1988), Trends in the development of baculovirus expression vectors. Bio/Technology 6, 47-55.
13. Ma, J J. et al. (1990), Purification and characterization of Saccharomyces cerevisiae uridine monophosphate kinase. Journal of Biol. Chem. 256 (31): 19122-19127.
14. Sambrook, J., Fritsch, E., F. and Maniatis, T. (1989), Molecular cloning: A laboratory manual. Cold spring harbor laboratory press, Cold spring harbor, New York.
15. Schulz, B., Banuett, F., Dahl, M., Schlesinger, R., Schäfer, W., Martin, T., Herskowitz, I. and Kahmann, R. (1990), The b alleles of Ustilago maydis, whose combinations program pathogenic development, code for polypeptides containing a homeodomain-related motif. Cell 60, 295-306.
16. Schultz etal. (1987), Gene 54:113-123.
17. Tsukuda, T. et al. (1988), Isolation and characterization of an autonomously replicating sequence from Ustilago maydis. Mol. Cell. Biol. 8: 3703-3709.
18. Yan, H. and Tsai, M D. (1999), Nucleoside monophosphate kinases: structure, mechanism, and substrate specificity. Adv. Enzymol. Relat. Areas Mol. Biol. 73:103-34.
19. Zhou, L. et al., (1998), Cloning, expression in Escherichia coli, and characterization of Arabidopsis thaliana UMP/CMP kinase. Plant Physiol. 117: 245-254.

1. A process for identifying fungicide compounds, comprising
(a) contacting a UMP/CMP kinase or a host cell that expresses a UMP/CMP kinase in sufficient quantity with a chemical compound or a mixture of chemical compounds under conditions that permit the interaction of the chemical compound with the polypeptide,
(b) comparing the activity of the UMP/CMP kinase in the absence of a chemical compound with the activity of the UMP/CMP kinase in the presence of a chemical compound or a mixture of chemical compounds, and
(c) ascertaining the chemical compound that specifically inhibits the UMP/CMP kinase.

2. A process according to claim 1, wherein the activity of the UMP/CMP kinase is determined by
(a) converting the ADP produced by the reaction of the UMP/CMP kinase to pyruvate and ATP by means of UMP/CMP kinase with the use of phosphoenolpyrivate,
(b) converting the pyruvate produced to lactate and NAD⁺ with lactate dehydrogenase using NADH, and
(c) determining the change of the NADH concentration.

3. A process according to claim 1 wherein a UMP/CMP kinase is used from a fungus that is pathogenic to plants.

4. A process according to claim 1 wherein in a further step the fungicide activity of the identified compound is tested by bringing it in contact with a fungus.

5. A process according to claim 2 wherein in a further step the fungicide activity of the identified compound is tested by bringing it in contact with a fungus.

6. A process to combat fungi that are pathogenic to plants, comprising allowing an inhibitor of a fungal UMP/CMP kinase to act on the fungus and/or its environment.

7. A nucleic acid coding for a UMP/CMP kinase from a fungus that is pathogenic to plants, comprising a sequence selected from:
a) a sequence according to SEQ ID NO: 1,
b) sequences that code for a polypeptide, that contains the amino acid sequence according to SEQ ID NO: 2,
c) sequences that code for a polypeptide that contains the motif [LIVMFYWCA]-[LIVMFYW](2)-D-G-[FYI]-P-R-x(3)-[NQ],
d) sequences that hybridize to the sequences defined under a) and b) at a hybridization temperature of 42 to 65° C., and
e) sequences which show at least an 80% sequence identity with the sequences defined under a) and b), preferred being those with at least 85% and particularly preferred those with at least a 90% identity.

8. A DNA construct comprising a nucleic acid according to claim 7 and a heterologous promoter.

9. A vector comprising a nucleic acid according to claim 7 or a DNA construct according to claim 8.

10. A vector according to claim 9, wherein the nucleic acid is functionally linked with regulating sequences that ensure the expression of the nucleic acid in pro- or eukaryotic cells.

11. A host cell comprising a nucleic acid according to claim 7, a DNA construct according to claim 8 or a vector according to claim 9 or 10.

12. A UMP/CMP kinase from fungi that are pathogenic to plants, comprising a sequence selected from:
(a) the sequence according to SEQ ID NO:2,
(b) sequences which show at least an 80% sequence identity with the sequence defined under a), preferred being those with at least 85%, particularly preferred those with at least 90% and especially preferred those with 95% identity,
(c) the sequences specified under b), which contain the motif [LIVMFYWCA]-[LIVMFYW](2)-D-G-[FYI]-P-R-x(3)-[NQ] and
(d) fragments of the sequences specified in a) through c), which show the same biological activity as the sequence defined under a).


SEQUENCE LISTING (2 sequences)

SEQ ID NO: 1 - DNA, 798 bp, Ustilago maydis
  CDS (1)..(798)
atgggtctgttcgacaagctcaagcacaagaagcacgaaccggtcgaggcagctcctgcccctgctgctcacattgcatctgcacccgctgctgatgctgagactggtgctggtgccgttgagaaggatacgccacgattcgactcgtccaaagtgacggtcgtctttgtgcttggcggacccggtgctggcaaaggcacccagtgtgctcgtctcgtacaagattacggctttgttcaccttagcgccggtgatctgctgcgtgccgaacagcaacgtccaggctcccagtacggtgccatgatcgccgactacatcaaggaaggcaagatcgtgcccatggaagtaaccgtcgctctgctttccaacgcgatcgcggaagctctctccaagcaagccactaccgaagccgatcactcgatccctgaagagcacaaactcaaatggagcgatggcaagggtcgcttccttgtcgacggcttcccacgcaagatggaccaggcgatcaaatttgacgaatcagtatgcgagtccaagtttgtgctgttcctgcagtgcagtgaagaagtgatgctcgagagattgctcgagcgtggaaagaccagcggtcgcgccgatgacaacatcgagtcgatcaagaagcgtttccagacctttgtagagacctcgatgcctgtggtcgactactaccgaaaacaggaccgcgtggtagaggtcgactcgatcaagacggtggatcaggtctatgccgagatcaaggaggctatggatcgcacctttgccaagcttgagcagtag

SEQ ID NO: 2 - protein, 265 aa, Ustilago maydis
MGLFDKLKHKKHEPVEAAPAPAAHIASAPAADAETGAGAVEKDTPRFDSSKVTVVFVLGGPGAGKGTQCARLVQDYGFVHLSAGDLLRAEQQRPGSQYGAMIADYIKEGKIVPMEVTVALLSNAIAEALSKQATTEADHSIPEEHKLKWSDGKGRFLVDGFPRKMDQAIKFDESVCESKFVLFLQCSEEVMLERLLERGKTSGRADDNIESIKKRFQTFVETSMPVVDYYRKQDRVVEVDSIKTVDQVYAEIKEAMDRTFAKLEQ